(19) United States
(12) Patent Application Publication
(10) Pub. No.: US 2004/0254487 A1
(43) Pub. Date: Dec. 16, 2004

(54) Traveling strip-chart marking control

(75) Inventors: Steven A. Mahoney, McMinnville, OR (US); Richard C. Myers, McMinnville, OR (US)

Correspondence Address: Robert D. Varitz, ROBERT D. VARITZ, P.C., 2007 S.E. Grant Street, Portland, OR, 97214, US

(73) Assignee: Inovise Medical, Inc.

(21) Appl. No.: 10/461,028

(22) Filed: Jun. 13, 2003

(51) Int. Cl. A61B 5/04

(52) U.S. Cl. 600/523

(57) ABSTRACT

A method and apparatus for controlling in a strip-chart recorder lateral and longitudinal imagery registration for overprinting on pre-printed heart imagery of visual pictorial, heart-related condition-interpretation data. Pairs of parallel and nonparallel control marks, precisely preprinted on a strip-chart page in relation to this pre-printed heart imagery, are observed passing through a prepared zone defined in the recorder. Near (or within) this prepared zone, a fixed-portion printhead, which is employed to perform overprinting, and whose print-active area can be laterally adjusted, is provided. Observation timings of passing control masks are employed to determine both (a) the times when data overprinting should occur, and (b) any necessary lateral printing-area adjustments that should be made, to assure proper printed-imagery registration.

### BACKGROUND AND SUMMARY OF THE INVENTION

This invention relates to print registration in a strip-chart recorder. In particular, it relates to a method and apparatus for controlling lateral and longitudinal registration of heart-related imagery over-printing (marking) onto pre-printed heart imagery that is provided on a strip-chart page traveling through a strip-chart recorder.

Two prior-issued U.S. Pat. Nos. 6,230,048 B1, and 6,516,220 B2, are hereby incorporated herein by reference to describe background for the present invention. In the practice described in those patents, a person's ECG information is examined in certain ways to provide an output interpretation data-signal that relates to the detection of particular, selected heart conditions. The output interpretation signal which is furnished is designed to enable and effect pictorial imaging of such conditions, in a manner which permits the incorporation, as by superimposing or printing, of this interpretation imagery in the visual setting of a normal heart structure. A very useful manner of presentation is illustrated in U.S. Pat. No. 6,516,220 B2, FIGS. 16A-16L. This manner involves the picturing of the whole heart structure as a kind of pseudo-three dimensional, exploded nominal assembly (in the sense of showing “normal” heart conditions) of regional slices, sufficiently separated so as to reveal internal heart structure, and to permit, with the application of interpretation superimposing or printing, an easy and very instructive way to visualize what the interpretation has “found”.

Clearly for such superimposed visual information to be reliably informative, its “placement” on, for example, pre-created (such as pre-printed) normal heart imagery must be very accurate. In other words, such superimposed, combined imagery must be done with a very accurate registration.

The present invention directly addresses this issue in the realm of printed (ECG-related) strip-chart pages (each a recording medium) which, according to the invention, are pre-printed with nominal, sliced, heart-regional imagery of the type mentioned above. These pages, separable, but initially end-attached to adjacent pages in a typical zigzag stack of pages, are fed through an otherwise conventional strip-chart data recorder which has been specially modified in accordance with the invention. In particular, the recorder has been modified to enable accurate overprinting of visual, pictorial representations of interpretation data developed as taught in the two, mentioned, prior U.S. Patents, which data is fed to appropriate input and image printing structure introduced to the recorder in the practice of the invention.

According to a preferred embodiment and manner of practicing the invention, elongate strip-chart pages are pre-printed with, in addition to the traditional time-based data-recording chart area, a region, containing precision-located, nominal, heart-slice imagery of the type earlier mentioned. These pages are also provided with groups, or pairs, of special linear control marks that lie in a certain manner adjacent one lateral edge of each page. These marks specifically lie at precision-established locations in relation to the preprinted heart imagery, and reside along a line which substantially parallels the long axis of the page. It is essentially along this long axis that a page travels through the recorder following what is referred to herein as a linear transport path in the recorder. The lines which form the mentioned marks are preferably straight-linear, and are organized, as will shortly be explained, with their long axes at certain angles of parallelism and non-parallelism relative to one another that play important roles in the practice of the invention. Specifically, time-based signals, that reflect observations of these lines as they pass a certain “reading” position within an observation zone defined in the recorder, provide the necessary lateral and longitudinal print-control information which is employed for the desired, highly accurate, imagery-overprinting registration information.

The recorder which cooperates in the practice of this invention is prepared with what is referred to as an observation zone which is equipped preferably with suitable electro-optical structure for observing the successive passages of the control marks that are provided, as stated earlier, on the prepared strip-chart pages. Also included in the recorder are a fixed-position printing agency which is elongate, and which extends laterally across, and preferably slightly laterally beyond, the lateral region in the recorder through which the full lateral extent of the preprinted heart imagery is nominally expected to pass. The printing agency, in accordance with the invention, possesses a laterally shiftable and reconfigurable lateral boundary capability.

Electronic print registration control structure is operatively interposed this printing agency and the control-mark observation structure, effectively to control both the proper timing for initiating an overprinting printing operation, as well as the exact lateral positioning which is to be employed for that operation.

The exact ways in which the specially prepared strip-chart medium and the recorder cooperatively interact to produce precisely positioned overprinting, as desired, will be more fully explained as the detailed description which now follows is read in conjunction with the accompanying drawings.

### DESCRIPTION OF THE DRAWINGS

FIG. 1A presents a simplified, schematic, side elevation of a strip-chart recorder made in accordance with a preferred and best mode embodiment of the present invention, shown in the process of feeding successive strip-chart pages, also made in accordance with the invention, through the recorder from a zigzag stack of yet un-recorded strip-chart pages.

FIG. 1B is an enlarged, fragmentary plan view taken very generally along line 1B-1B in FIG. 1A.

FIG. 2 is an even more enlarged and stylized schematic plan view of the preferred arrangement of print registration control marks that are pre-printed along one side of each prepared strip-chart page.

FIG. 3 is a fragmentary plan view, on about the same scale employed in FIG. 1B, further illustrating the operative relationship which exists, according to the invention, between a strip-chart page, and observation and controlled printing structure present in the recorder.

FIG. 4 is a fragmentary plan view, on a larger scale than that employed in FIGS. 1 and 2, illustrating a portion of a strip-chart page which has been over-printed in accordance with the invention.

FIG. 5 is an isolated detail drawn from FIG. 4 illustrating in dotted-outline form interpretation-data overprinting which is similar to that shown in FIG. 4.

FIG. 6 is a block/schematic structural and functional diagram of the system of the invention.

FIG. 7 is a schematic circuit diagram of the “SENSOR” block shown in FIG. 6.

### DETAILED DESCRIPTION OF THE DRAWINGS

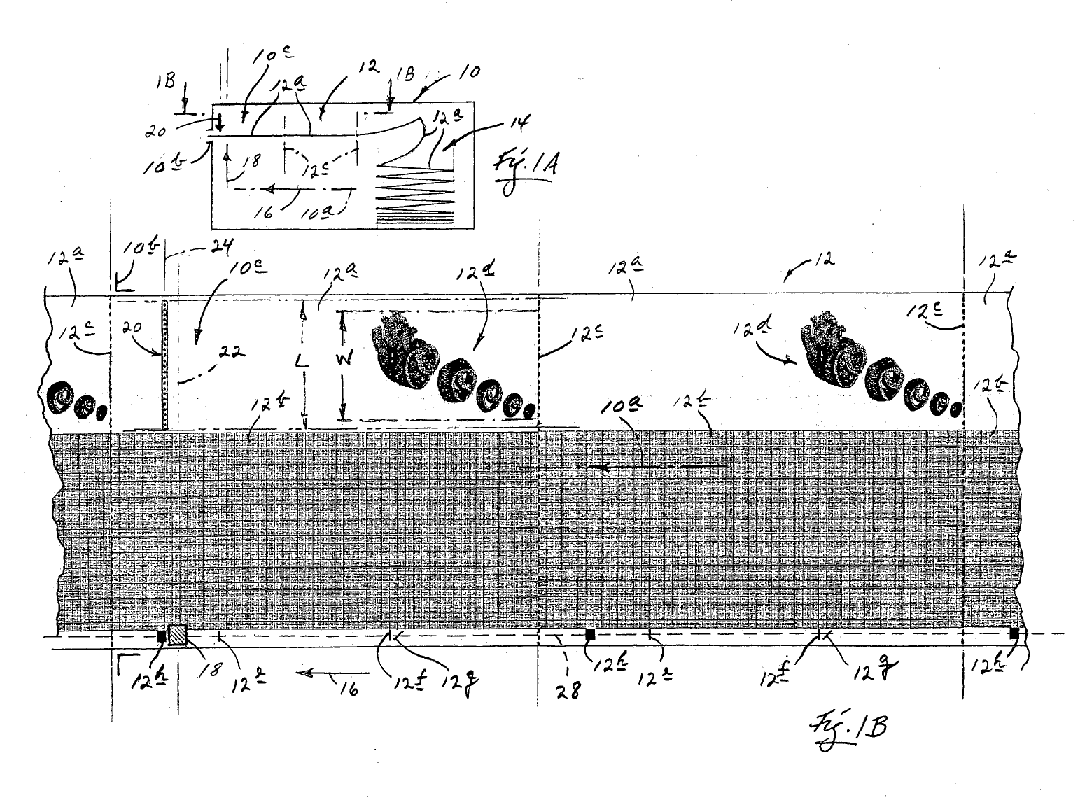

Turning now to the drawings, and referring initially to FIGS. 1-3, inclusive, FIGS. 1A, 1B generally illustrate a strip-chart printing recorder 10, and a strip-chart recording medium 12, which are made, and which perform, in accordance with a preferred and best-mode embodiment of, and manner of practicing, the present invention. Recorder 10 draws successive, end-to-end, separably connected strip-chart pages 12a from a conventional zigzag, or Z-fold, stack 14 of these pages, and thru-feeds/transports such pages along a linear transport path 10a in the direction of arrow 16. In FIG. 1A, the right side, or end, of recorder 10 is the upstream side, and the left side, or end, is the downstream side. This downstream side includes a strip-chart page discharge port 10b. Suitable structure is furnished adjacent this port to accommodate easy tear-free separation of printed chart pages. Such tear-separation is accommodated by conventional pre-perforated tear lines located at end regions between adjacent pages, shown at 12c.

Utilizing conventional printing structure and associated electrical circuitry (not shown) which forms part of recorder 10, traditional ECG wave data is printed on a conventional time-base graph, grid, or chart, such as that shown at 12b. This graph portion of each page 12a is conventional, and not part of the present invention. However, specially included within recorder 10, either by way of original-manufacture construction, or by way of retrofitting if desired, is an observation zone 10c which, as illustrated in the invention embodiment now being described, is adjacent recorder discharge port 10b. Located within zone 10c are an electro-optical sensor, or observation structure, 18, and slightly downstream therefrom, an elongate, fixed-position linear print, or printing, agency 20. As can be seen, these two structures are simply represented by two arrows in FIG. 1A.

Sensor 18 looks down upon the upwardly facing surface of strip-chart pages, and lies on a transverse line shown at 22 in FIGS. 1B and 3. Print agency, hereinafter referred to as “printhead”, 20 extends along another transverse line shown at 24 in FIGS. 1B and 3. Lines 22, 24 substantially parallel one another, and are disposed substantially orthogonally with respect to transport path 10a. The spacing between these two lines as measured in the direction of path 10a is precisely established, known, and is relevant to proper functioning of the present invention. Any suitable spacing may be used.

Printhead 20 may be entirely conventional in construction, and herein takes the form of a slender, linear row of ink-jet nozzles coupled to suitable and conventionally configured printing support structure (not shown). The length L of printhead 20, as shown in FIG. 1B, is seen to be greater than the established width W of the overall lateral, or transverse, dimension of a pre-printed sliced-heart visual imagery group 12d which resides at the prepared-area location shown on each strip-chart page 12a. In the embodiment of the invention now being described, printhead 20 has a length L which is slightly in excess of about 2.5-inches a dimension W is slightly less than about 2.5-inches.

When a strip-chart 12a is traveling along path 10a through recorder 10, its transverse dimension between its lateral edges, generally speaking, is substantially centered on this path. With that situation in existence, the lateral, transverse boundaries of pre-printed area 12d lie within the extremes of dimension L in FIG. 1B. However, because of the fact that a certain amount of tolerenced lateral play (for anti-binding clearance purposes) is permitted the width of strip-chart document within recorder 10, it is possible for the strip-chart medium to shift in one direction or another laterally, whereby its longitudinal centerline is no longer laterally aligned with path 10a. It is to accommodate this lateral misalignment situation that the overall length L of printhead 20 is made somewhat larger than the lateral width W of pre-printed heart group 12d.

Referring for a moment especially to FIG. 3 wherein printhead 20 is given an exaggerated appearance, this printhead possesses what is referred to herein as a laterally shiftable and reconfigurable boundary for its operative printing activity area (pointed to generally by arrow 20A), and the lateral extremes of this boundary are shown in FIG. 3. Very specifically, the solid outline rectangle 20a represents a shift in the printing activity area of printhead 20 upwardly in FIG. 3 to one of its lateral limits. Dashed lines 20b illustrate opposite-direction shifting to the other lateral limit condition for this printing activity area. This reconfigurable and shiftable boundary capability which is provided printhead 20 in accordance with the invention is one of the features that allows accurate print registration for overprinting to occur throughout the possible total range of lateral drift, or misalignment, in both directions which might occur for a strip-chart page relative to path 10a.

The nominal, lateral centerline which is shared by imagery group 12d and printhead 20 when a page 12a is centered on path 10a is shown at 25 in FIG. 3.

Also provided by way of pre-preparation application on and along one edge of each strip-chart page, in accordance with practice of the present invention, are special, elongate, straight-linear control marks, such as the marks shown at 12e, 12f, 12g. As can be seen especially well in FIG. 1B, mark 12e (one for each chart page) is relatively close to the left ends of the two, whole strip-chart pages which are pictured in this figure. Marks 12f, 12g are disposed closely adjacent one another and more toward the right ends of these same strip-chart pages. A block-like, somewhat square mark 12h, referred to herein as a stop-control mark, is located on each page to the left of mark 12e, as seen in FIG. 3.

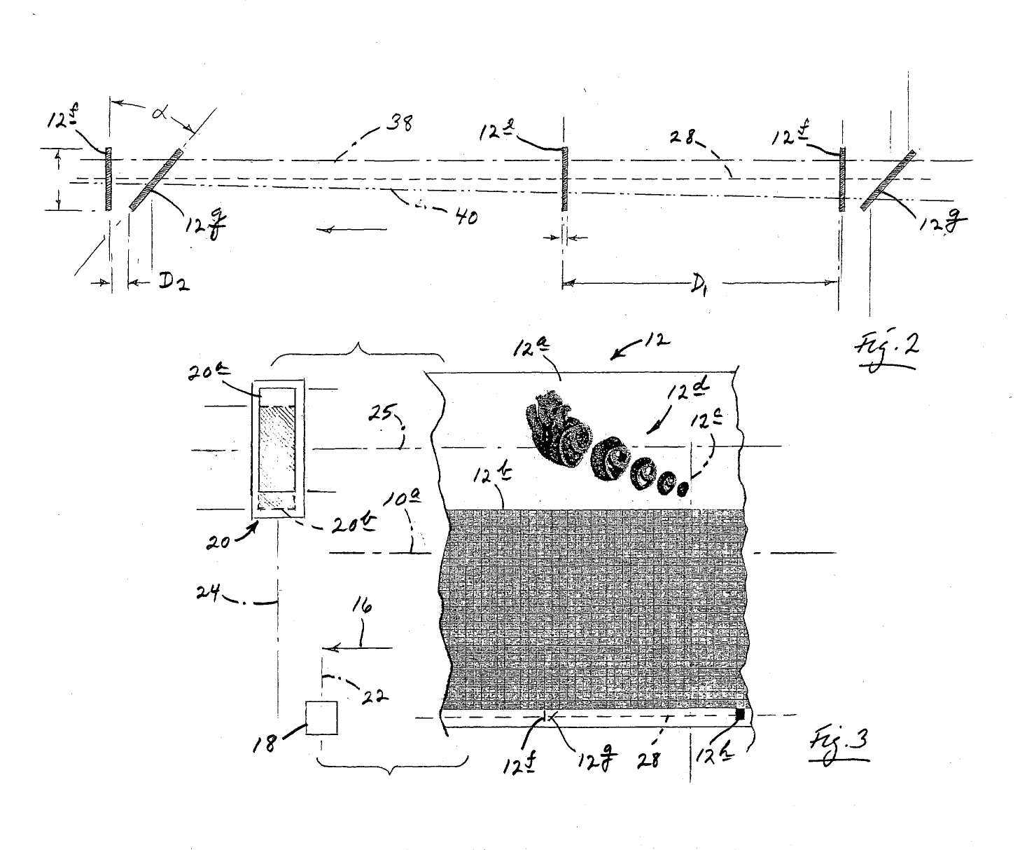

Looking now particularly at FIG. 2 in the drawings, here the several control marks prepared in accordance with the invention are shown in an enlarged, stylized and isolated fashion to aid in an understanding of the preferred structures and positionings of these marks. On each page, marks 12e, 12f are spaced apart by a distance D₁ of about 110-millimeters, are elongate, and are disposed with their long axes substantially parallel to one another. These two marks' long axes are also substantially parallel to previously mentioned lines 22, 24 which are substantially at right angles to transport path 10a. Control mark 12g is disposed at an angle of about 45-degrees relative to its next-adjacent, neighboring mark 12f, with that end of mark 12g which is closest to its neighboring mark 12f being spaced therefrom by a distance D₂ of about 3-millimeters. Each of these marks has an overall length of about 8-millimeters, and a width of about 1.25-millimeters. Within a given strip-chart page, marks 12e, 12f are referred to herein as being one pair of parallel-related marks, and marks 12f, 12g as being another pair of nonparallel-related marks.

As can be seen in FIGS. 1B, 2 and 3, marks 12e, 12f, 12g reside along the lower edge of each strip-chart page 12a, with all of these marks lying substantially laterally centered on and along a line shown as a dashed line 28. Line 28 substantially parallels the long axis of each strip-chart page, and thus nominally also substantially parallels transport path 10a when the associated chart-page is traveling through recorder 10. Marks 12e, 12f, 12g, 12h are sufficiently darkened so as to be clearly “readable”, or observable, as each of them passes in succession beneath electro-optical sensor 18 during transport of a strip-chart page through the recorder.

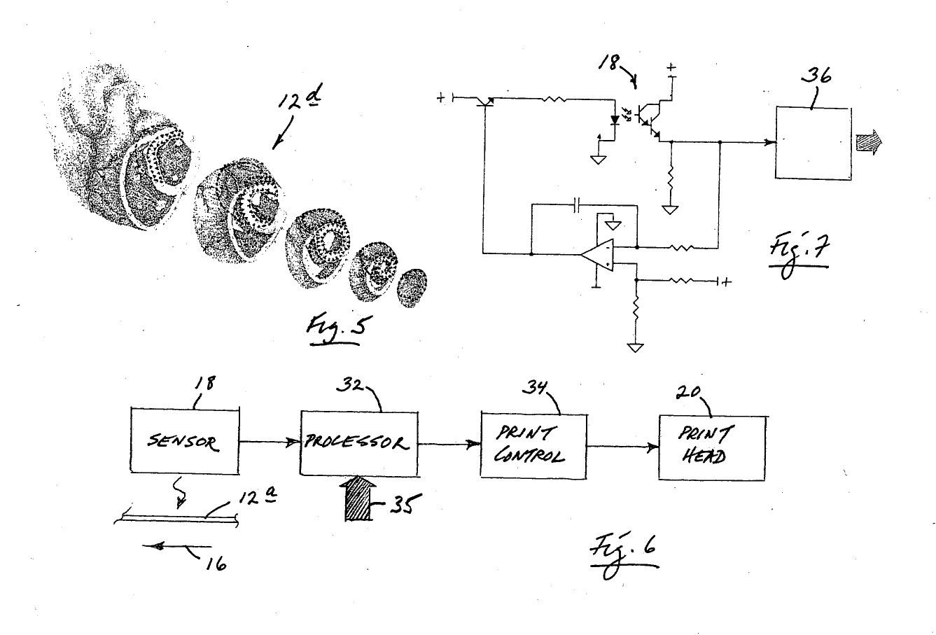

Turning attention now to especially to FIGS. 6 and 7, and further describing components of the present invention, sensor 18 feeds a digital output signal to a data processor 32 which assesses the information contained in this sensor output signal to control the operation of printhead 20 through a block 34 labeled “PRINT CONTROL” in FIG. 6. An interpretation output signal of the kind mentioned earlier in relation to the two referenced U.S. patents is supplied effectively to processor 32 as indicated by broad, shaded data arrow 35 in FIG. 6. Processor 32 and print control 34 collectively constitute what is referred to herein both as part of a print-alignment structure, and as print registration control structure. A preferred form for the operative structure of sensor 18 is shown in detail in the circuit/block diagram presented in FIG. 7. The componentry shown in FIG. 7, connected as shown therein, fully defines the operative structure of a preferred form of sensor 18 whose direct analog output signal is fed to an analog-to-digital converter 36. It is the digital output from converter 36 which is fed to previously mentioned processor 32. The circuitry presented in FIG. 7 is believed to be entirely self-explanatory, and one will note that the circuitry pictured there produces an operating electro-optical sensor capable of operating within a linear operating range.

Explaining now the operation of this invention, just prior to initiating the transport of a page 12a through the recorder, that page “sits” essentially in a stopped position as is illustrated for the left-hand full page shown in FIG. 1B. In this position, one will note that the associated stop-control mark 12b has “previously” just passed beneath sensor 18, and the page has appropriately stopped. When page transport begins, as the page now travels through recorder 10 to record input ECG wave data as a plot on the chart portion of the page, control marks 12e, 12f, 12g pass in succession beneath sensor 18. This passage in succession of these marks relative to sensor 18 is referred to herein as defining, or being, a predetermined-characteristic relationship which, as will be seen, is a time relationship. The sensor produces for each such passage an output signal, typically a pulse, and it is the timing information that relates to the time spacings of control mark observations (these pluses) that is employed in the practice of the invention to control accurate overprinting activity of the type earlier mentioned herein.

Very specifically, with the distance between parallel-paired marks 12e, 12f, known, that information, along with the time duration noted between observations of the passages of these two marks by sensor 18, gives information relative to the then transport speed of the subject strip-chart page. This information controls the timing for operation of printhead 20 which, effectively, is fed (as mentioned above) an interpretation output signal of the type described in the earlier-referenced two United States patents. Thus, it is the cooperative interaction of control marks 12e, 12f and sensor 18 that plays the role of responsibility in controlling longitudinal registration accuracy, that is, longitudinal with respect to the long axis of a strip-chart page which, of course, substantially parallels transport path 10a.

Depending upon the lateral position which a strip-chart page possesses as it travels through the recorder and through observation zone 10c, the timing existing between observations of the passages of nonparallel-paired marks 12f, 12g relative to sensor 18 is directly relevant to the lateral position (alignment/misalignment) then occupied by the associated strip-chart page. Thus, it is this timing information which is employed to adjust, if necessary, the lateral position of print activity area 20A of printhead 20, thus to control precise lateral positioning of an overprinting operation.

Looking for a moment again especially at FIG. 2, when marks 12e, 12f, 12g travel past sensor 18 with line 28 exactly laterally centered under the sensor, the timing which is observed between the passages of marks 12f, 12g gives an indication that the associated pre-printed heart-image group 12d will pass laterally centered relative to printhead 20. In this situation, no lateral boundary shifting of the printhead's effective printing activity area is needed.

If, however, there is a parallel, lateral misalignment that exists between line 28 and the lateral centerline of sensor 18, such as is illustrated by dash-dot line 38 in FIG. 2, then a lateral adjustment is required of the printhead's print activity area. The line-38-type misalignment means that line 38 will effectively occupy the same plane (normal to the plane of FIG. 2) as does the lateral centerline of sensor 18, and a printhead “boundary shift” will be produced in the direction of the adjustment of activity area 20A pictured at 20b in FIG. 3. Obviously, if such a parallel misalignment occurs in the opposite direction, boundary shifting will occur with activity area 20A moving in the direction of the adjustment pictured at 20a in FIG. 3.

Similar boundary shifts are produced to take care of slight skew misalignments, such as the skew misalignment suggested by dash-double-dot line 40 in FIG. 2. By equipping the system with an appropriate memory structure, not specifically shown herein, and by keeping a record of the recent passages of pairs of nonparallel control marks 12f 12g, it is also possible to use comparative timing information to assess a transport skew condition which might exist in the transport of strip-chart pages.

Still with reference to FIG. 2, when no lateral misalignment of any kind exists, then the timing information relevant to determining transport speed is the time “marked” by the transit distance D₁, and that relevant to determining whether printing-activity-area boundary shifting is required is “marked” by transit distance D₃. With a parallel, lateral misalignment in existence, as suggested by line 38, transit distance D₁, remains that which is relevant to determining transport speed, but a new distance D₄ now “marks” transit activity which is relevant to area-20A boundary shifting. In a skew situation, such as that suggested by line 40, timing information now relevant to determining transport speed is the time “marked” by the transit distance shown at D₅, and boundary-shift information relating to area 20A is marked by transit distance D₆.

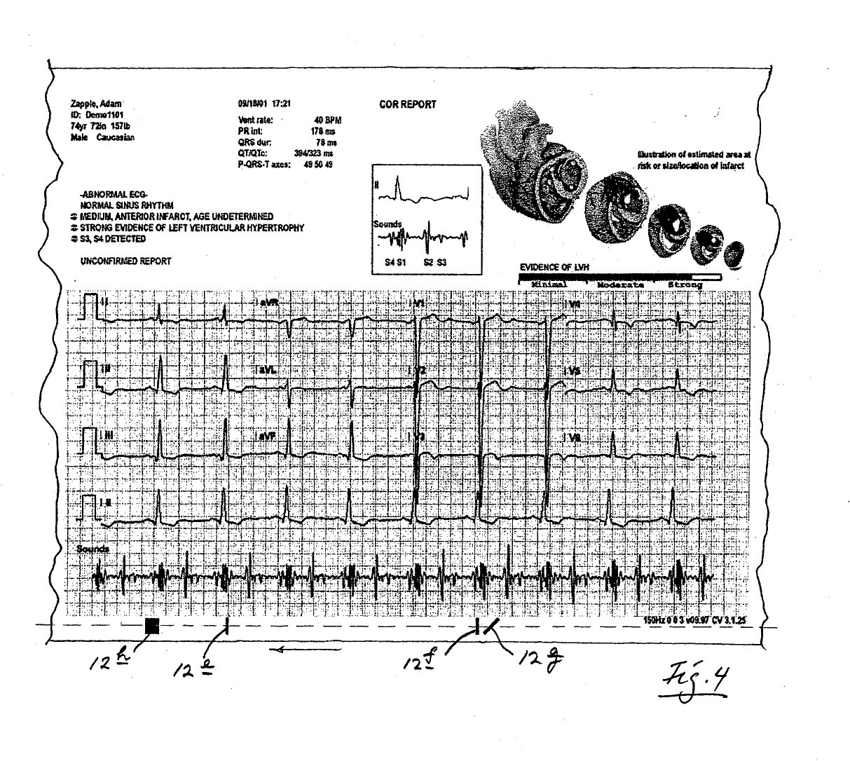

FIG. 4 in the drawings, along with FIG. 5, provide two different illustrations of a properly overprinted and registered display of visual imagery derived from an interpretation output signal of the type earlier described with respect to the operation of printhead 20. In FIG. 4, which illustrates a fragment of a completely printed strip-chart page, the several noticeably darker regions provided within the sliced heart preprinted imagery 12d represents overprinting of information drawn from that interpretation output signal. FIG. 5 illustrates this very same general kind of overprinting condition, but here, from another illustrative point of view, shows overprinting areas in the form of dotted outlines.

It should thus be apparent that the system and methodology proposed by the present invention offer a unique way to effect precision longitudinal and lateral registration control for the overprinting of heart-related interpretation data of the type described and illustrated herein. Changes in strip-chart transport speed, skewing of a chart, and lateral shifting of a chart page within recorder 10 are easily converted into timing signals produced by observations of the passages, proposed by the invention, of the several control marks relative to sensor 18 to effect precise lateral and longitudinal overprinting registration. Stop-control marks 12h, when observed passing beneath sensor 18, cause the system of the invention, as mentioned, to stop the motion of strip-chart page transport at the end of each per-page overprinting and waveform, printing operation.

Accordingly, while a preferred embodiment and manner of practicing the invention have been illustrated and described herein, it is appreciated that variations and modifications may be made without departing from the sprit of the invention.

We claim:

1. A method for controlling lateral and longitudinal print registration in the environment of a strip-chart recorder through which a chart medium on which printing therein is to occur travels along a defined, longitudinally extending, linear transport path comprising
providing plural, optically observable control marks on such a medium at spaced locations distributed thereon along a line which, with medium traveling through the recorder, generally substantially parallels the mentioned transport path,
observing the successive passages of such marks as each passes in the recorder a defined observation zone associated with longitudinal transport of the medium through the recorder, and
utilizing a predetermined-characteristic relationship between selected, successive, observed passages of such marks relative to the mentioned observation zone to control both lateral and longitudinal print registration.

2. The method of claim 1, wherein the predetermined-characteristic relationship is a time relationship.

3. The method of claim 1, wherein said providing includes the applying of elongate marks which are linear in nature, and with respect to which a first pair of such spaced, applied marks includes two marks that are substantially parallel to one another, and second pair of such marks includes two marks that are nonparallel to one another.

4. The method of claim 2, wherein said providing includes the applying of elongate marks which are linear in nature, and with respect to which a first pair of such spaced, applied marks includes two marks that are substantially parallel to one another, and a second pair of such marks includes two marks that are nonparallel to one another.

5. The method of claim 1 which further effects, in addition to lateral and longitudinal print-registration control, skew registration control.

6. The method to claim 5, wherein the predetermined-characteristic relationship is a time relationship.

7. The method of claim 5, wherein said providing includes the applying of elongate marks which are linear in nature, and with respect to which a first pair of such spaced, applied marks includes two marks that are substantially parallel to one another, and second pair of such marks includes two marks that are nonparallel to one another.

8. The method of claim 6, wherein said providing includes the applying of elongate marks which are linear in nature, and with respect to which a first pair of such spaced, applied marks includes two marks that are substantially parallel to one another, and second pair of such marks includes two marks that are nonparallel to one another.

9. The method of claim 1, wherein printing for which registration control is implemented is performed by a fixed-position printing agency having a selectively boundary-shiftable and reconfigurable printing activity area, and print registration control is performed by managing boundary selection of the printing agency's printing activity area in accordance with said utilizing step.

10. The method of claim 9, wherein the predetermined-characteristic relationship is a time relationship.

11. The method of claim 9, wherein said providing includes the applying of elongate marks which are linear in nature, and with respect to which a first pair of such spaced, applied marks includes two marks that are substantially parallel to one another, and second pair of such marks includes two marks that are nonparallel to one another.

12. The method of claim 9 which further effects, in addition to lateral and longitudinal print-registration control, skew registration control.

13. The method of claim 12, wherein the predetermined-characteristic relationship is a time relationship.

14. The method of claim 12, wherein said providing includes the applying of elongate marks which are linear in nature, and with respect to which a first pair of such spaced, applied marks includes two marks that are substantially parallel to one another, and second pair of such marks includes two marks that are nonparallel to one another.

15. The method of claim 1, wherein the chart medium takes the form of an elongate chart page which enters the strip-chart recording environment, which chart page is detachably end-connected to at least one next-adjacent, elongate chart page.

16. An elongate, print-registration-ready, strip-chart recording medium which is intended for transport along, and with its long axis generally paralleling, a linear travel path defined through a strip-chart recorder which includes an observation zone and a printing zone, said medium comprising
plural, optically observable control marks resident at plural locations on a surface in said medium, spaced and distributed along a line which substantially parallels the medium's said long axis,
said control marks being generally straight-linear, and taking the form of (a) a first pair of marks including marks that are substantially parallel to one another, and (b) a second pair of marks including marks that are nonparallel to one another.

17. Chart-medium print-alignment structure interactively and operatively associating a printable chart medium and a strip-chart printing recorder through which such a chart medium travels along a defined, longitudinally extending, linear transport path during interactive operation of the structure, and in which recorder printing is to take place on such a chart medium, said structure comprising
plural, optically observable control marks provided on such a medium at spaced locations distributed thereon along a line which, with medium traveling through the recorder, generally substantially parallels the mentioned transport path,
an observation zone in the recorder through which the medium travels during transport through the recorder, including structure for observing the successive passages of such marks moving through the observation zone, and
print registration control structure operatively connected to said observing structure, operable to utilize a predetermined-characteristic relationship between selected, successive, observed passages of such marks relative to the mentioned observing structure to control both lateral and longitudinal registration of printing on the medium.

18. The print-alignment structure of claim 17, wherein the mentioned predetermined-characteristic relationship is a time relationship.

19. The print-alignment structure of claim 17, wherein said elongate marks are linear in nature, and include a first pair of spaced marks characterized by two marks that are substantially parallel to one another, and second pair of such marks characterized by two marks that are nonparallel to one another.

20. The print alignment structure of claim 17, wherein said recorder further includes, for printing on a chart medium, a fixed-position printing agency having a selectively boundary-shiftable and reconfigurable printing activity area, said print registration control structure is operatively connected to said printing agency, and print registration control is implemented over the printing agency by the print registration control structure through the action of managing boundary selection of the printing agency's printing activity area in relation to the operation of said observing structure.

21. A method employable in a strip-chart recording device, and in relation to an elongate, defined-boundary strip-chart recording medium having lead and trailing end regions, and spaced lateral edges extending between such end regions, which medium is moved, during recording, along a defined travel path through a recording zone in the device, for controlling the registration of visual makings which are to be applied in the recording zone as surface overprinting to a prepared area of preprinted visual imagery disposed at a known location relative to the medium's end regions and lateral edges, said method comprising,
preparing the medium with plural, longitudinally spaced, machine-observable markings which are generally placed on the medium along a nominal line which, during transport of the medium through the recording zone, is intended generally to parallel the mentioned, defined travel path,
observing the successive passages of such markings as each passes in the recorder a defined observation zone associated with longitudinal transport of the medium through the recorder, and
utilizing a predetermined-characteristic relationship between selected, successive, observed passages of such markings relative to the mentioned observation zone to control both lateral and longitudinal print registration.
